(19) United States
(12) Patent Application Publication
(10) Pub. No.: US 2014/0219999 A1
(43) Pub. Date: Aug. 7, 2014

(54) TREATMENT OF COLON CANCER USING COMPLEMENT INHIBITORS

(71) Applicants: John D. Lambris, Philadelphia, PA (US); Joseph Nadeau, Seattle, WA (US); Stephanie Doerner, Shaker Heights, OH (US); Nathan A. Berger, Shaker Heights, OH (US)

(72) Inventors: John D. Lambris, Philadelphia, PA (US); Joseph Nadeau, Seattle, WA (US); Stephanie Doerner, Shaker Heights, OH (US); Nathan A. Berger, Shaker Heights, OH (US)

(73) Assignee: The Trustees of the University of Pennsylvania, Philadelphia, PA (US)

(21) Appl. No.: 14/008,074

(22) PCT Filed: Mar. 30, 2012
(86) PCT No.: PCT/US12/31461

§ 371 Date: Mar. 31, 2014

Related U.S. Application Data
(60) Provisional application No. 61/470,674, filed on Apr. 1, 2011.

(51) Int. Cl. A61K 38/17; C07K 16/28

(52) U.S. Cl. CPC A61K 38/17; C07K 16/28
USPC 424/133.1; 514/19.3

(57) ABSTRACT

Methods for treating, preventing or delaying onset of polyp formation and subsequent colon cancer are disclosed. The methods involve administration of a complement inhibitor to inhibit C5a receptor signaling in the tumorigenic or pre-tumorigenic tissue.

Pursuant to 35 U.S.C. §202(c), it is acknowledged that the United States government may have certain rights in the invention described herein, which was made in part with funds from the National Institutes of Health under Grant Nos. GM-62134 and AI-068730.

### FIELD OF THE INVENTION

This invention relates to the field of oncology and cancer therapy. Methods for treating, preventing or delaying onset of formation of colon cancer are provided. The methods involve administration of a complement inhibitor to inhibit C5a receptor signaling in the treatment or prevention of colon cancer.

### BACKGROUND OF THE INVENTION

Various publications, including patents, published applications, technical articles and scholarly articles are cited throughout the specification. Each of these cited publications is incorporated by reference herein, in its entirety. Full citations for publications not cited fully within the specification are set forth at the end of the specification.

Colon cancer is the third most common cancer worldwide and is the second leading cause of cancer related deaths in the United States. Both environmental and genetic factors such as diet, obesity, microbial profile, inflammatory diseases and heritability are believed to contribute to susceptibility. However, the strong correlation between obesity and diet in humans makes it difficult to evaluate the independent contributions that each make to the development and progression of colon cancer.

There is evidence that associates chronic inflammation and colon cancer. Links between inflammation and colon cancer have been shown epidemiologically by showing an increased risk of developing colon cancer in patients with inflammatory bowel disease, ulcerative colitis and Crohn's disease as well as the effectiveness of anti-inflammatory drugs in the reduction of intestinal tumor formation. Obesity, which can increase risk of colon cancer development, is associated with chronic inflammation and the release of pro-inflammatory cytokines, such as tumor necrosis factor-α (TNF-α), interleukin-6 (IL-6) and IL-1β. These cytokines are thought to be involved in inflammatory bowel disease as well as colon cancer (Pischon et al., 2008, Proc Nutr Soc. 67: 128-145; Fantini & Pallone, 2008, Curr Drug Targets 9: 375-380; Old, 1985, Science 230: 630-632; Mudter & Neurath, 2007, Inflamm. Bowel Dis. 13: 1016-1023; van Heel et al., 2002, Hum. Mol. Genet. 11: 1281-1289). Interleukin-6 (IL-6) is a pro-inflammatory cytokine that is secreted by T-cells and macrophages to stimulate an immune response. IL-6 is also produced by adipocytes and high levels increase anti-apoptotic factors such as Bcl-2 and Bcl-xl in humans and increase tumor number and size in mice (Fantini & Pallone, 2008, supra).

The innate immune system is a type of immunity that has many functions in the body including the induction of cytokine production for the recruitment of immune cells to sites of infection, identification of foreign pathogens to induce an immune response, and the activation of the complement component system. The complement component system is a biochemical cascade involved in both the innate immune system and the activation of the adaptive immune system. The complement component system is important for the identification and clearance of foreign pathogens that involves both classical, alternative and mannose-binding lectin pathways. Complement component 3 (C3) is central to the activation of all pathways, is critical to the cleavage and subsequent activation of the potent chemoattractant, complement component C5a (C5a), and is responsible for mediating the inflammatory response during infection (Carroll, 2008, Vaccine 26S: 128-133).

Ablation of C5a in mouse models of chemically induced colitis reduced the release of proinflammatory cytokines and inhibited the infiltration of neutrophils in colon tissue (Guojiang, 2010, Laboratory Investigation advance online publication, Nov. 22, 2010 1-12). Inflammatory diseases (inflammatory bowel disease, ulcerative colitis, Crohn's disease) of the intestine are associated with an increased risk of developing colon cancer in humans and studies have shown that complement component C3 is elevated in patients with these diseases (Lundgren et al., 2010, Eur. J. Gastroenterol. Hepatol. 22: 429-436).

Recent research has shown that ablation of C3 or the C5a receptor can decrease tumor growth in mouse models (Markiewski et al., 2008, Nat. Immunol. 9: 1225-1235). Complement component C3 and C5a can activate the protein kinase AKT via phosphatidylinositol-3-kinase (PI3K). The targets of the PI3K/AKT pathway have biological functions, many are essential for cancer growth and development. These biological processes include cell survival, cell-cycle progression, cell growth and cell metabolism. Glycogen synthase kinase-3 (GSK-3) is regulated by AKT and controls a number of important cell-cycle events by phosphorylation of cell-cycle regulators (c-Myc and cyclin DO and transcription factors that govern cell fate and differentiation (c-Jun, (β-catenin, Notch). AKT regulates cell survival by the inactivation the pro-apoptotic protein BAD and activation of the IκB kinase (IKK), a key component of the nuclear factor-κB (NFκB) pathway.

Despite investigation into the anti-cancer potential of the complement system, a distinct role of the complement system in colon cancer has not been elucidated, due at least to the complex interactions among diet, obesity, inflammation and immunity in this disease. Advances in the art are needed to provide a practical link between the complement system and its modulation for the purpose of controlling or treating colon cancer.

### SUMMARY OF THE INVENTION

One aspect of the invention features a method for treating or preventing colon cancer in an individual. The method comprises administering a therapeutically effective amount of a complement inhibitor to the individual, wherein the complement inhibitor reduces or prevents C5a receptor signaling in the tumorigenic or pre-tumorigenic tissue, thereby preventing, reducing or delaying one or more of polyp formation, polyp growth and polyp tumorigenesis. The method is particularly suitable for human subjects, though not limited to humans.

The complement inhibitor can include one or more of a C5a inhibitor, a C5aR inhibitor, a C3 inhibitor, a C3aR inhibitor, a factor D inhibitor, a factor B inhibitor, a C4 inhibitor, a C1q inhibitor, or any combination thereof. In one embodiment, the complement inhibitor is a C5a inhibitor or a C5aR inhibitor. Examples include acetyl-Phe-[Orn-Pro-D-cyclohexylalanine-Trp-Arg] (PMX-53), PMX-53 analogs, neutrazumab, TNX-558, eculizumab, pexelizumab or ARC1905, or any combination thereof. In another embodiment, the complement inhibitor is a C3 inhibitor. Examples include compstatin, a compstatin analog, a compstatin peptidomimetic, a compstatin derivative, or any combinations thereof. Particular examples include SEQ ID NO:1, SEQ ID NO:2, SEQ ID NO:3 or SEQ ID NO:4. In another embodiment, the complement inhibitor is a C4 inhibitor.

The complement inhibitor can be administered or targeted to the colon, or it can be administered systemically. In various embodiments, the complement inhibitor is administered together or concurrently with, or sequentially before or after, at least one other colon cancer treatment. It can also be administered as a solo treatment.

Another aspect of the invention features a pharmaceutical composition for the treatment or prevention of colon cancer. The pharmaceutical composition comprises one or more complement inhibitors and at least one colon cancer treatment agent, in a pharmaceutically acceptable medium.

The complement inhibitor can include one or more of a C5a inhibitor, a C5aR inhibitor, a C3 inhibitor, a C3aR inhibitor, a factor D inhibitor, a factor B inhibitor, a C4 inhibitor, a C1q inhibitor, or any combination thereof. In one embodiment, the complement inhibitor is a C5a inhibitor or a C5aR inhibitor. Examples include acetyl-Phe-[Orn-Pro-D-cyclohexylalanine-Trp-Arg] (PMX-53), PMX-53 analogs, neutrazumab, TNX-558, eculizumab, pexelizumab or ARC1905, or any combination thereof. In another embodiment, the complement inhibitor is a C3 inhibitor. Examples include compstatin, a compstatin analog, a compstatin peptidomimetic, a compstatin derivative, or any combinations thereof. Particular examples include SEQ ID NO:1, SEQ ID NO:2, SEQ ID NO:3 or SEQ ID NO:4. In another embodiment, the complement inhibitor is a C4 inhibitor.

The pharmaceutical composition can be formulated for administration targeted to the colon, or it can be formulated for systemic administration. In one embodiment, the composition is formulated for oral administration, which can encompass both systemic and targeted administration.

Another aspect of the invention features a method of reducing inflammation in the gastrointestinal system of an individual. The method comprises administering a therapeutically effective amount of a complement inhibitor to the individual, wherein the complement inhibitor reduces or prevents accumulation of one or more proinflammatory molecules in the individual's gastrointestinal system, thereby reducing inflammation. In one embodiment, the method is used to reduce inflammation in the colon of the individual. In particular embodiments, the proinflammatory molecules are activated AKT, IKK, NFκB or any combination thereof.

Other features and advantages of the invention will be understood by reference to the drawings, detailed description and examples that follow.

### BRIEF DESCRIPTION OF THE DRAWINGS

FIG. 1. Construction of the chromosome substitution strains (CSSs) and consomic-congenic strains (CCSs). A: B6 and A/J are crossed to produce F1 progeny. Offspring are chosen that contain the desired A/J chromosome and these mice are mated to B6 in order to remove all other A/J chromosomes from the background. After 10 or more generations of mating, mice that are heterozygous only for the chromosome of interest are chosen. These mice are intercrossed to produce a mouse that is homosomic for the desired chromosome, thereby completing the CSS strain. B: CSSs strains that are resistant or susceptible to diet-induced obesity were used to construct congenic-consomic strains for our studies. They were constructed by crossing the CSS with the A/J chromosome of interest to B6.Apc^{Min/+} heterosomic. The F1 progeny that were heterozygous for Apc^{Min/+} were backcrossed to the original CSS to obtain a homozygous A/J chromosome.

FIG. 2. Chromosome Substitution Strains (CSSs) were chosen to construct Congenic-Consomic Strains (CCSs) based on consistent, contrasting responses to diet-induced obesity. After 100 days on a high fat diet, B6, B6.A2, B6.A9 and B6.A19 were obese, while A/J, B6.A7 and B6.A17 remained lean. These strains were used to generate CCSs that were predisposed to intestinal cancer, yet susceptible or resistant to diet-induced obesity.

FIG. 3. Polyp numbers are significantly reduced in A2.Apc^{Min/+} compared to B6.Apc^{Min/+}, regardless of diet. There is a significant reduction in the total polyp number of A2.Apc^{Min/+} after 60 days on the LF_{Coco} and HF_{Coco} diets when compared to B6.Apc^{Min/+}.

FIG. 4. Complement component C3 is elevated in B6.Apc^{Min/+} after 30 and 60 days on the HF_{Coco} diet. Serum was collected via the orbital sinus from B6.Apc^{Min/+} and B6 littermates after 30 and 60 days on the LF_{Coco} and HF_{Coco} diets and used to measure circulating levels of complement component C3. C3 is significantly increased in B6.Apc^{Min/+} fed the HF_{Coco}.

FIG. 5. Complement C5a is elevated in B6.Apc^{Min/+} fed a HF diet. Circulating complement component C5a was measured by ELISA from serum extracted from the orbital sinus. After 30 and 60 days on the HF_{Coco} diet, C5a was significantly increased B6.Apc^{Min/+} (blue) compared to B6 littermates fed the same diet. Circulating C5a was not detectable in A2.Apc^{Min/+} (purple) after 60 days on the HF_{Coco} diet.

FIG. 6. Mice deficient in C3aR have significant reduction in polyp number and size. B6.Apc^{Min/+} were crossed with C3aR knockout mice to generate mice susceptible to intestinal neoplasia that were also deficient in one or two copies of C3a receptor. C3aR−/− mice had a significant reduction in polyp number and cross sectional polyp area, after 30 days on the HF_{Coco} diet. Data plotted as Average±SEM.

FIG. 7. C5aR inhibitor significantly reduces polyp numbers and area. A. After 30 days of treatment, a significant reduction in polyp number was observed in B6.Apc^{Min/+} treated with the C5aR inhibitor (purple) compared to untreated (blue) or mice treated with a control inhibitor (red). B. Cross sectional polyp area was also significantly reduced in mice treated with the C5aR inhibitor. Data plotted as Average±SEM.

FIG. 8. Pharmacological inhibition of C5aR reduces NFκB signaling in the intestine after 30 days on the HF_{Coco} diet. Mice treated with the C5aR inhibitor for 30 days had a significant reduction in the phosphorylation of AKT, IKK and NFκB compared to mice treated with the control inhibitor.

### DETAILED DESCRIPTION OF ILLUSTRATIVE EMBODIMENTS

Various terms relating to the methods and other aspects of the present invention are used throughout the specification and claims. Such terms are to be given their ordinary meaning in the art unless otherwise indicated. Other specifically defined terms are to be construed in a manner consistent with the definition provided herein.

### DEFINITIONS

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, and nucleic acid chemistry and hybridization are those well known and commonly employed in the art.

Standard techniques are used for nucleic acid and peptide synthesis. The techniques and procedures are generally performed according to conventional methods in the art and various general references (e.g., Ausubel et al., 2011, Current Protocols in Molecular Biology, John Wiley & Sons, NY), which are provided throughout this document.

The nomenclature used herein and the laboratory procedures used in analytical chemistry and organic syntheses described below are those well known and commonly employed in the art. Standard techniques or modifications thereof, are used for chemical syntheses and chemical analyses.

As used herein, each of the following terms has the meaning associated with it in this section.

The singular form of a word includes the plural, and vice versa, unless the context clearly dictates otherwise. Thus, the references “a”, “an”, and “the” are generally inclusive of the plurals of the respective terms. For example, reference to “a compound” or “a method” includes a plurality of such “compounds” or “methods.” Similarly, the words “comprise”, “comprises”, and “comprising” are to be interpreted inclusively rather than exclusively. Likewise the terms “include”, “including” and “or” should all be construed to be inclusive, unless such a construction is clearly prohibited from the context.

The terms “comprising” or “including” are intended to include embodiments encompassed by the terms “consisting essentially of” and “consisting of”. Similarly, the term “consisting essentially of” is intended to include embodiments encompassed by the term “consisting of”.

Dosages expressed herein are in units per kilogram of body weight (e.g., μg/kg or mg/kg) unless expressed otherwise.

Ranges are used herein in shorthand, to avoid having to list and describe each and every value within the range. Any appropriate value within the range is intended to be included in the present invention, as is the lower terminus and upper terminus, independent of each other.

The term “about” as used herein when referring to a measurable value such as an amount, a temporal duration, and the like, is meant to encompass variations of ±20% or ±10%, in some embodiments ±5%, in some embodiments ±1%, and in some embodiments ±0.1% from the specified value, as such variations are appropriate to practice the disclosed methods or to make and used the disclosed compounds, compositions or articles of manufacture.

The term “antibody” refers to an immunoglobulin molecule that is able to bind specifically to a particular epitope on an antigen. Antibodies can be intact immunoglobulins derived from natural sources or from recombinant sources and can be immunoreactive portions of intact immunoglobulins. The antibodies useful in the present invention may exist in a variety of forms including, for example, polyclonal antibodies, monoclonal antibodies, intracellular antibodies (“intrabodies”), Fv, Fab and F(ab)₂, as well as single chain antibodies (scFv), camelid antibodies and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

A “complement inhibitor” is a molecule that prevents or reduces activation and/or propagation of the complement cascade that results in the formation of C3a or signaling through the C3a receptor, or C5a or signaling through the C5a receptor. A complement inhibitor can operate on one or more of the complement pathways, i.e., classical, alternative or lectin pathway. A “C3 inhibitor” is a molecule or substance that prevents or reduces the cleavage of C3 into C3a and C3b. A “C5a inhibitor” is a molecule or substance that prevents or reduces the activity of C5a. A “C5aR inhibitor” is a molecule or substance that prevents or reduces the binding of C5a to the C5a receptor. A “C3 aR inhibitor” is a molecule or substance that prevents or reduces binding of C3a to the C3a receptor. A “factor D inhibitor” is a molecule or substance that prevents or reduces the activity of Factor D. A “factor inhibitor” is a molecule or substance that prevents or reduces the activity of factor B. A “C4 inhibitor” is a molecule or substance that prevents or reduces the cleavage of C4 into C4b and C4a. A “C1q inhibitor” is a molecule or substance that prevents or reduces C1q binding to antibody-antigen complexes, virions, infected cells, or other molecules to which C1q binds to initiate complement activation. Any of the complement inhibitors described herein may comprise antibodies or antibody fragments, as would be understood by the person of skill in the art.

Colorectal cancer, or colon cancer, usually begins as a polyp. The word “polyp” is a nonspecific term to describe an outgrowth on the internal lining of the colon. Common types of polyps found in the large intestine include hyperplastic polyps and adenomatous polyps (or adenomas), both of which may proceed to frank malignancy (cancer), although this most commonly occurs with the adenomatous polyps. While polyps may be detected as benign, premalignant (potentially malignant) growths, virtually all colorectal cancer develops from these benign growths. Consequently, surgical or pharmacologic intervention to remove or interfere with polyp progression is an important component of colorectal cancer prevention.

A “subject”, “individual” or “patient” refers to an animal of any species. In various embodiments, the animal is a mammal. In one embodiment, the mammal is a human. In another embodiment, the mammal is a non-human animal.

“Treating” refers to any indicia of success in the treatment or amelioration of the disease or condition. Treating can include, for example, reducing or alleviating the severity of one or more symptoms of the disease or condition, or it can include reducing the frequency with which symptoms of a disease, defect, disorder, or adverse condition, and the like, are experienced by a patient. “Treating” can also refer to reducing or eliminating a condition of a part of the body, such as a cell, tissue or bodily fluid, e.g., blood. “Preventing” refers to the partial or complete prevention of the disease or condition in an individual or in a population, or in a part of the body, such as a cell, tissue or bodily fluid (e.g., blood). The term “prevention” does not establish a requirement for complete prevention of a disease or condition in the entirety of the treated population of individuals or cells, tissues or fluids of individuals. The term “treat or prevent” is sometimes used herein to refer to a method that results in some level of treatment or amelioration of the disease or condition, and contemplates a range of results directed to that end, including but not restricted to prevention of the condition entirely. Thus, in the treatment or prevention of colorectal cancer, the skilled artisan will understand that treatment can be administered at several stages of disease progression. Further, treatment at an early stage, such as the stage of polyp formation, can reduce or prevent the incidence of colon cancer.

A “prophylactic” treatment is a treatment administered to a subject (or sample) that does not exhibit signs of a disease or condition, or exhibits only early signs of the disease or condition, for the purpose of decreasing the risk of developing pathology associated with the disease or condition. For instance, treatment of an individual at risk of colon cancer (due to family history, previous development of polyps, and/or various environmental or lifestyle risk factors) can be considered a prophylactic treatment designed to reduce, delay or prevent later development of colon cancer. This term may be used interchangeably with the term “preventing,” again with the understanding that such prophylactic treatment or “prevention” does not establish a requirement for complete prevention of a disease in the entirety of the treated population of individuals or tissues, cells or bodily fluids.

As used herein, a “therapeutically effective amount” or simply an “effective amount” is the amount of a composition sufficient to provide a beneficial effect to the individual to whom the composition is administered, or who is otherwise treated using a method involving the composition.

### DESCRIPTION

The invention springs in part from the inventors' clear demonstration that complement deficiencies and pharmacological blockade of the C5a receptor reduces polyp formation in an animal model of colon cancer that is predictive of the human condition. Pharmacological inhibition of C5a receptor-mediated signaling resulted in significant reduction in activated AKT serine-threonine protein kinase (pS473), IkB kinas (IKK) (pS176) and NFκB (pS536) mRNA and/or protein levels.

The discoveries made in accordance with the present invention provide support for the utility of a novel therapeutic option, complement inhibition, in the treatment or prevention of colon cancer. This utility is particularly advantageous because of the relatively small number of side effects reported for complement-directed therapy (Kohl et al., 2006, Curr. Opin. Mol. Ther. 8: 529-538; Ricklin et al., 2007, Nature Biotechnol. 25:1265-1275), as compared with the high toxicity associated with currently used anti-cancer chemotherapeutics.

The findings that support certain aspects of the invention, set forth in the Examples herein, indicate that the complement system and particularly C5a contribute to mechanisms that promote polyp formation. Generally, the activation of C5 requires prior activation of C3 (Sahu et al., 2001, Immunol. Rev. 180: 35-48). However, under specific pathophysiological conditions, C5a can be generated in the absence of C3 (Huber-Lang et al., 2006, Nature Med. 12:682-687). Therefore, the similar degree of inhibition of polyp incidence and/or growth that was observed in C3-deficient and C5aR antagonist-treated mice indicates that C5 activation may require prior cleavage of C3. Accordingly, aspects of the present invention encompass inhibition of C5a receptor mediated signaling not only at the receptor, but at any point in the complement activation cascade leading to the production of C5a.

One aspect of the invention provides a method for treating or preventing colorectal tumor formation in an individual. Specifically, the method comprises administering a complement inhibitor, as described in greater detail below.

In certain embodiments, complement inhibitors are used for reducing the incidence and/or aggressiveness (e.g., growth rate) of polyp formation, as exemplified herein. Reducing polyp formation and/or growth concomitantly reduces the likelihood of tumorigenesis from the polyp tissue. This prophylactic treatment is suitable for individuals at risk of developing colon cancer, as assessed by various known risk factors (family history, physical condition, diet, exercise, or an inflammatory disease or condition of the colon, to name a few). It is also suitable for individuals who have exhibited a propensity for developing polyps in the past, and have had them removed. In other embodiments, complement inhibitors are used as a treatment of colon cancer itself. In still other embodiments, complement inhibitors are administered as part of a follow-up or post-treatment of a patient who has been or is being treated for colon cancer, e.g., by surgery, chemotherapy or a combination. Thus, the timing or time frame in which the complement inhibitors are administered will depend on the stage of disease progression being treated, as would be readily apparent to the skilled artisan.

Because of the localization of the tumors in the gastrointestinal tract, certain embodiments feature targeted delivery of the complement inhibitor to the site of the tumor, i.e., through oral or rectal administration, or by instrument-assisted deposition of the inhibitor into the colon. However, in the animal model described in the examples, the complement inhibitor was injected at a site away from the colon.

Targeted delivery to the tumor may be accomplished by physical targeting, e.g., by injection or deposition at the tumor site, or into the colon orally or rectally, or by chemical or biological targeting, e.g., by linking or associating the complement inhibitor with an agent that has an affinity for the tumor, such as an anti-tumor cell antibody. However, targeted delivery is not believed to be required or preferred in all cases.

Any inhibitor of C5a formation or activity may be used in the method of the invention. Inhibition of C5a formation or activity may be accomplished in a variety of ways. For instance, C5a activity may be inhibited directly by preventing or significantly reducing the binding of C5a to its receptor, C5aR. A number of C5aR inhibitors are known in the art. Acetyl-Phe-[Orn-Pro-D-cyclohexylalanine-Trp-Arg] (AcF[OPdChaWR]; PMX-53; Peptech) is a small cyclic hexapeptide that is a C5aR antagonist and is exemplified herein. Analogs of PMX-53 (e.g., PMX-201 and PMX-205) that also function as C5aR antagonists are also available (see for instance Proctor et al., 2006, Adv Exp Med. Biol. 586:329-45 and U.S. Pat. Pub. No. 20060217530). Neutrazumab (G2 Therapies) binds to C5aR, thereby inhibiting binding of C5a to C5aR. Neutrazumab (G2 Therapies) binds to extracellular loops of C5aR and thereby inhibits the binding of C5a to C5aR. TNX-558 (Tanox) is an antibody that neutralized C5a by binding to C5a.

C5a activity may also be inhibited by reducing or preventing the formation of C5a. Thus, inhibition of any step in the complement cascade which contributes to the downstream formation of C5a is expected to be effective in practicing the invention. Formation of C5a may be inhibited directly by inhibiting the cleavage of C5 by C5-convertase. Eculizumab (Alexion Pharmaceuticals, Cheshire, Conn.) is an anti-C5 antibody that binds to C5 and prevents its cleavage into C5a and C5b. Pexelizumab, a scFv fragment of Eculizumab, has the same activity. Similarly, ARC1905 (Archemix), an anti-C5 aptamer, binds to and inhibits cleavage of C5, inhibiting the generation of C5b and C5a.

In another embodiment, formation of C5a is reduced or prevented through the use of a C3 inhibitor. Preferably, the C3 inhibitor is Compstatin or a Compstatin analog, derivative, aptamer or peptidomimetic. Compstatin is a small molecular weight cyclic peptide having the sequence Ile-Cys-Val-Val-Gln-Asp-Trp-Gly-His-His-Arg-Cys-Thr (SEQ ID NO. 1). Examples of Compstatin analogs, derivatives and peptidomimetics are described in the art. See, for instance, U.S. Pat. No. 6,319,897, U.S. Pat. No. 7,888,323, WO/1999/013899, WO/2004/026328 and WO/2010/127336.

An exemplary Compstatin analog comprises a peptide having a sequence: Xaa1-Cys-Val-Xaa2-Gln-Asp-Trp-Gly Xaa3-His-Arg-Cys-Xaa4 (SEQ ID NO. 2); wherein:

Xaa1 is Ile, Val, Leu, Ac-Ile, Ac-Val, Ac-Leu or a dipeptide comprising Gly-Ile;

Xaa2 is Trp or a peptidic or non-peptidic analog of Trp;

Xaa3 is His, Ala, Phe or Trp;

Xaa4 is L-Thr, D-Thr, Ile, Val, Gly, or a tripeptide comprising Thr-Ala-Asn, wherein a carboxy terminal OH of any of the L-Thr, D-Thr, Ile, Val, Gly or Asn optionally is replaced by —NH₂; and the two Cys residues are joined by a disulfide bond. Xaa1 may be acetylated, for instance, Ac-Ile. Xaa2 may be a Trp analog comprising a substituted or unsubstituted aromatic ring component. Non-limiting examples include 2-naphthylalanine, 1-naphthylalanine, 2-indanylglycine carboxylic acid, dihydrotryptophan or benzoylphenylalanine.

Another exemplary Compstatin analog comprises a peptide having a sequence: Xaa1-Cys-Val-Xaa2-Gln-Asp Xaa3-Gly-Xaa4-His-Arg-Cys-Xaa5 (SEQ ID NO. 3); wherein:

Xaa1 is Ile, Val, Leu, Ac-Ile, Ac-Val, Ac-Leu or a dipeptide comprising Gly-Ile;

Xaa2 is Trp or an analog of Trp, wherein the analog of Trp has increased hydrophobic character as compared with Trp, with the proviso that, if Xaa3 is Trp, Xaa2 is the analog of Trp;

Xaa3 is Trp or an analog of Trp comprising a chemical modification to its indole ring wherein the chemical modification increases the hydrogen bond potential of the indole ring;

Xaa4 is His, Ala, Phe or Trp;

Xaa5 is L-Thr, D-Thr, Ile, Val, Gly, a dipeptide comprising Thr-Asn or Thr-Ala, or a tripeptide comprising Thr-Ala-Asn, wherein a carboxy terminal OH of any of the L-Thr, D-Thr, Ile, Val, Gly or Asn optionally is replaced by NH₂; and the two Cys residues are joined by a disulfide bond. The analog of Trp of Xaa2 may be a halogenated trpytophan, such as 5-fluoro-1-tryptophan or 6-fluoro-1-tryptophan. The Trp analog at Xaa2 may comprise a lower alkoxy or lower alkyl substituent at the 5 position, e.g., 5-methoxytryptophan or 5-methyltryptophan. In other embodiments, the Trp analog at Xaa 2 comprises a lower alkyl or a lower alkenoyl substituent at the 1 position, with exemplary embodiments comprising 1-methyltryptophan or 1-formyltryptophan. In other embodiments, the analog of Trp of Xaa3 is a halogenated tryptophan such as 5-fluoro-1-tryptophan or 6-fluoro-1-tryptophan.

An exemplary Compstatin analog of this type is Ac—I[CVW(Me)QDWGAHRCT]I—NH₂ (SEQ ID NO:4), which can be synthesized as described by Katragadda M, et al., 2006, J Med Chem. 49: 4616-4622.

Another set of exemplary Compstatin analogs features Compstatin or any of the foregoing analogs, in which Gly at position 8 is modified to constrain the backbone conformation at that location. In one embodiment, the backbone is constrained by replacing the Gly at position 8 (Gly8) with Nα-methyl Gly.

Other C3 inhibitors include vaccinia virus complement control protein (VCP) and antibodies that specifically bind C3 and prevent its cleavage.

In other embodiments, formation of C5a is reduced or prevented through the use of an inhibitor of complement activation prior to C3 cleavage, e.g., in the classical or lectin pathways of complement activation. Non-limiting examples of such inhibitors include, but are not limited to: (1) factor D inhibitors such as diisopropyl fluorophosphates and TNX-234 (Tanox), (2) factor B inhibitors such as the anti-B antibody TA106 (Taligen Therapeutics), (3) C4 inhibitors (e.g., anti-C4 antibodies) and (4) C1q inhibitors (e.g., anti-C1q antibodies). Likewise, inhibitors of signaling via the C3a receptor are also contemplated as being useful in the present invention.

Antibodies useful in the present invention, such as antibodies that specifically bind to either C4, C3 or C5 and prevent cleavage, or antibodies that specifically bind to factor D, factor B, C1q, or the C3a or C5a receptor, can be made by the skilled artisan using methods known in the art. See, for instance, Harlow, et al. (1988, In: Antibodies, A Laboratory Manual, Cold Spring Harbor, N.Y.), Tuszynski et al. (1988, Blood, 72:109-115), U.S. patent publication 2003/0224490, Queen et al. (U.S. Pat. No. 6,180,370), Wright et al., (1992, Critical Rev. in Immunol. 12(3,4):125-168), Gu et al. (1997, Thrombosis and Hematocyst 77(4):755-759) and Burton et al., (1994, Adv. Immunol. 57:191-280). Anti-C3 and anti-C5 antibodies are also commercially available.

The invention encompasses the use of pharmaceutical compositions comprising a complement inhibitor to practice the methods of the invention. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single-or multi-does unit.

As used herein, the term “pharmaceutically-acceptable carrier” means a chemical composition with which a complement inhibitor may be combined and which, following the combination, can be used to administer the complement inhibitor to a mammal

As used herein, the term “physiologically acceptable” ester or salt means an ester or salt form of the active ingredient which is compatible with any other ingredients of the pharmaceutical composition, which is not deleterious to the subject to which the composition is to be administered.

The pharmaceutical compositions useful for practicing the invention may be administered to deliver a dose of between 1 ng/kg/day and 100 mg/kg/day. In one embodiment, the invention envisions administration of a dose which results in a concentration of a complement inhibitor between 1 μM and 10 uM in an individual diagnosed with or at risk of developing colon cancer. While the precise dosage administered will vary depending upon any number of factors, including but not limited to, the type of patient and type of disease state being treated, the age of the patient and the route of administration. Preferably, the dosage of the compound will vary from about 1 mg to about 10 g per kilogram of body weight of the patient. More preferably, the dosage will vary from about 10 mg to about 1 g per kilogram of body weight of the patient.

The pharmaceutical composition may be administered to a patient as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the patient, as described above.

A single complement inhibitor may be administered or two or more different complement inhibitors may be administered in the practice of the method of the invention. In one embodiment of the invention, the method comprises administration of only a complement inhibitor. In other embodiments, other biologically active agents are administered in addition to the complement inhibitor in the method of the invention. Non-limiting examples of other biologically active agents useful in the invention include various classes of chemotherapy drugs, including platinum complexes (such as carboplatin), mitotic inhibitors, alkylating agents, antimetabolites, antitumor antibiotics and DNA topoisomerase inhibitors (referred to collectively herein as “anti-cancer agents”). Where feasible, the complement inhibitors may be combined with one or more other anti-cancer agents into a single pharmaceutical composition. Alternatively, separate pharmaceutical compositions are utilized.

As mentioned above, pharmaceutical compositions comprising the complement inhibitor may be administered before, during, and/or after another treatment of the precipitating illness or condition, such as surgery, radiation therapy or treatment with another anti-cancer agent. Chemotherapeutic agents commonly used, both alone and in various combinations, for the treatment of colorectal cancer include but are not limited to 5-fluoruracil, Leucovorin, Irinotecan, Capecitabine and Oxaliplatin. Monocolonal antibodies, such as Bevacizumab, Cetuximab and Panitumumab, are also used either alone or along with chemotherapy for treatment of colorectal cancer.

Pharmaceutical compositions that are useful in the methods of the invention may be administered systemically in oral solid formulations, parenteral, ophthalmic, suppository, aerosol, topical or other similar formulations. Such pharmaceutical compositions may contain pharmaceutically-acceptable carriers and other ingredients known to enhance and facilitate drug administration. Other formulations, such as nanoparticles, liposomes, resealed erythrocytes, and immunologically based systems may also be used to administer a complement inhibitor according to the methods of the invention.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intravenous, subcutaneous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, in microbubbles for ultrasound-released delivery or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

As used herein, “additional ingredients” include, but are not limited to, one or more of the following: excipients; surface active agents including replacement pulmonary surfactants; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” that may be included in the pharmaceutical compositions of the invention are known in the art and described, for example in Genaro, ed., 1985, Remington's Pharmaceutical Sciences, Mack Publishing Co., Easton, Pa., which is incorporated herein by reference.

The pharmaceutical compositions comprising complement inhibitors and/or other active agents or additional ingredients, can be conveniently packaged together in kits. Such kits comprise at least the complement inhibitor and instructions for its use in treating or preventing colon cancer. Such kits may also comprise the complement inhibitor and another anti-cancer agent, along with instructions for their use in treating colon cancer. The kits may also comprise one or more of the diluents, excipients, carriers and other ingredients referred to above. They may also comprise reagents and other components for diagnosing or detecting the stages of cancer, such as reagents to detect biomarkers of cancer progression.

The following examples are provided to describe the invention in greater detail. They are intended to illustrate, not to limit, the invention.

### Example 1

This example describes animals and diets utilized in the subsequent examples.

Mouse Models

Two mouse models were combined to create a novel model system. The first model was an intestinal cancer susceptible strain, B6.Apc^{Min/+} (Min=Multiple Intestinal Neoplasia), that closely resembles aspects of the human disease. The second was a set of strains, the Chromosome Substitution Strains (CSSs), which have contrasting responses to a high fat diet. These CSSs were used to construct congenic-consomic strains (CCSs) that are susceptible to colon cancer, but are either resistant or susceptible to diet-induced obesity, depending on the CSS.

B6.Apc^{Min/+} is a mouse model for intestinal neoplasia that carry a mutation in the Apc gene similar to that found in over 85% of sporadic colon cancer cases in humans. The APC mutation results in improper binding of β-catenin to the degradation complex and models the mutation observed in most sporadic human colon cancer tumors. B6.Apc^{Min/+} mice develop 50-80 intestinal polyps in the small intestine by 120 days of age (Heijstek et al., 2005, Digest Surg. 22: 16-25). The APC amino acid sequence is 90% identical to that of the human APC.

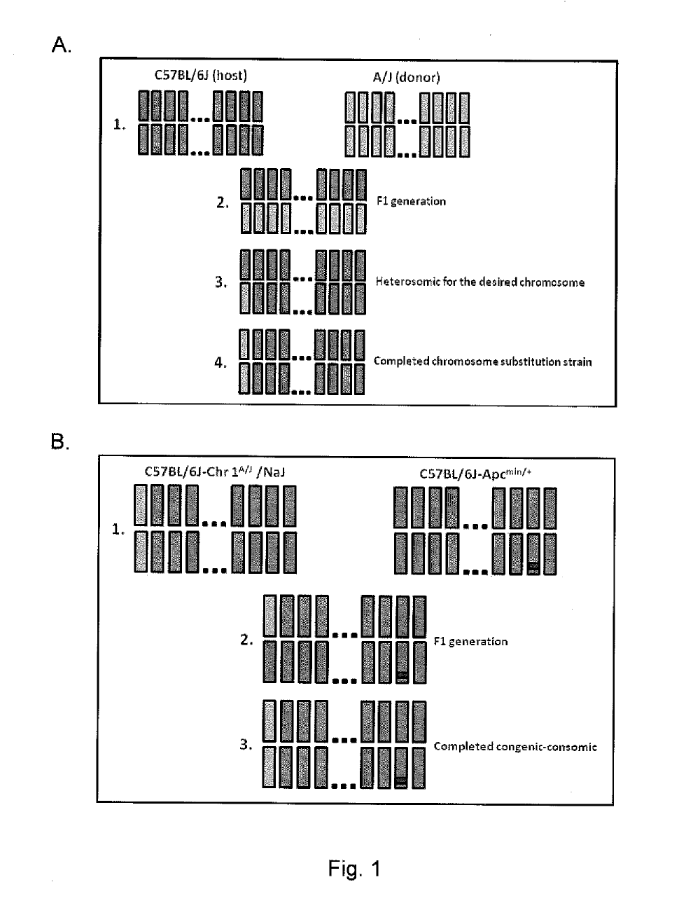

Chromosome Substitution Strains (CSSs) are made by replacing a chromosome of a donor strain (A/J) with the corresponding chromosome of a host strain (B6) (Nadeau et al., 2000, Nat. Genet. 24: 221-225; Singer et al., 2004, Science 304: 445-448) (FIG. 1). The F1 progeny that result from this initial cross are then backcrossed to the parental B6 strain. For 10 or more generations, progeny are chosen that have the A/J chromosome of interest. This ensures that remaining A/J DNA segments are lost through subsequent crossings and results in a fixed B6 background. After 10 or more generations, mice are heterosomic for the A/J chromosome of interest, whereas all other chromosomes are homosomic for B6. These mice are then intercrossed to obtain mice that are homosomic for the A/J chromosome of interest.

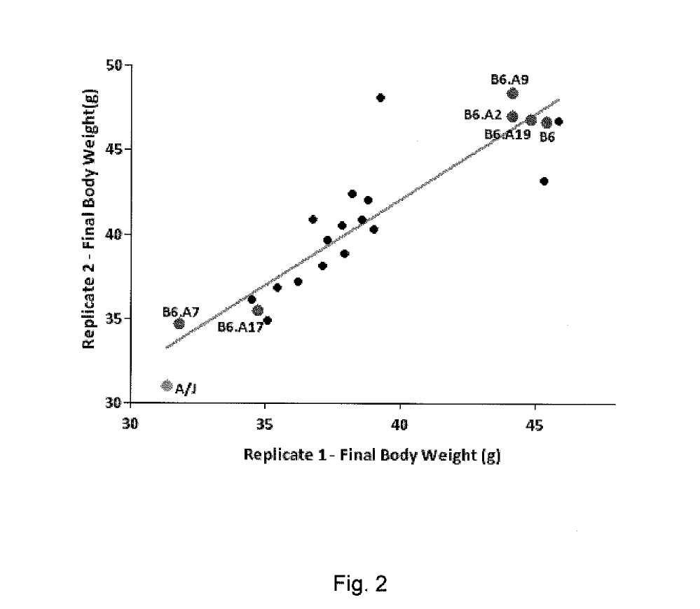

Certain of the Congenic-Consomic strains (CCSs) were used to separate the independent contributions of diet and obesity on polyp number, location and size. CSSs that were chosen for the construction of the CCSs based on contrasting responses to dietary fat (FIG. 2), whereby some strains were consistently lean (B6.A7, B6.A17) and others fat (B6.A2, B6.A9, B6.A19) after 100 days on a high fat diet. To create CCSs that were susceptible to intestinal polyps, B6.Apc^{Min/+} were crossed to CSSs that differed in susceptibility to diet-induced obesity (FIG. 2). B6.Apc^{Min/+} were mated with a CSS of interest and the F1 progeny backcrossed to the original CSS strain to obtain a homosomic A/J chromosome. Because the APC mutation is embryonic lethal, only mice heterozygous for the mutation can be obtained.

Several chromosomes contain known modifiers that alter the polyp frequency in B6.Apc^{min/+}. Chromosomes (genes) 4(Mom1), 8(Foxl1), and 18(Mom2, Mom3, Mom7) that have been shown to have these modifiers were avoided when selecting the CSSs. Modifiers have not been identified on the chromosome chosen for the studies. Although precautions were taken to avoid introducing complications by using chromosome without known modifiers, it is still possible that unknown modifiers exist on these substituted chromosomes.

B6.A2, B6.A7, B6.A9, B6.A17, and B6.A19 are homosomic for A/J chromosomes 2, 7, 9, 17 and 19, respectively, whereas the remainder of the genetic background is derived from B6. When these strains are crossed to B6.Apc^{Min/+}, the CCSs that result (A7.Apc^{Min/+} and A17.Apc^{Min/+}) are resistant to diet-induced obesity, but susceptible to colon cancer. Of these, three strains were chosen (B6.A2, B6.A9, and B6.A19) to create CCSs that are susceptible to diet-induced obesity (A2.Apc^{Min/+}, A9.Apc^{Min/+}, and A19.Apc^{Min/+}). Because B6.A7 and B6.A17 are resistant to diet-induced obesity, they could be used to test the effects of diet, independent of obesity, on cancer severity.

B6.C3aR^{-/-} are deficient in the complement C3 receptor (C3aR) and will be used to test interactions between dietary fatty acids and the complement component system. C3aR knockout mice have reduced TNF-α production, neutrophil infiltration, mast cell degranulation, and increased susceptibility to infection by Group B streptococci. C3aR deficient mice (B6.C3aR^{-/-}) will be crossed to mice heterozygous for the APC mutation (B6.Apc^{Min/+}) to generate mice that are susceptible to colon cancer and lack a crucial factor in the complement component pathway (C3aR^{-/-}; Apc^{Min/+}). The F1 progeny that have the APC mutation will be crossed to heterozygous B6.C3aR^{+/-} to generate test (C3aR^{+/-}; Apc^{Min/+}, C3aR^{-/-}; Apc^{Min/+}) and control (C3aR^{+/+}; Apc^{+/+}, C3aR^{+/-}; Apc^{+/+}, C3aR^{-/-}; Apc^{+/+}, C3aR^{+/+}; Apc^{Min/+}) mice for the diet studies described below.

Diets

Diets were formulated to differ in the amount of fat, but to be identical in vitamins, protein sources, and minerals (Table 1). Two diets contained 58% kcal/g fat (HF) from hydrogenated coconut oil (saturated fat) or corn oil (omega-6 polyunsaturated fat). Low fat diets were used as controls that contain 10.5% kcal/g fat (LF) also from hydrogenated coconut oil or corn oil. The amount of carbohydrates was increased in the LF diet to compensate for the loss of calories from the fat. For this reason, the HF and LF diets had comparable caloric values with 5558.5 kcal/g and 5557.0 kcal/g, respectively. Four diets were constructed in total (two test, two control) that differed in the kind and amount of fat (Table 1). The hydrogenated coconut oil diet (HF_{Coco}) is high in saturated fat (99.1%) and the majority of the fatty acids are derived from lauric, myristic and stearic acids. The corn oil diet (HF_{corn}) is high in omega-6 polyunsaturated fatty acids and the majority of the fatty acids are derived from linoleic, oleic and palmitic acids.

TABLE 1
General composition of diets used for diet studies.
All HF diets contained 58% kcal of fat per gram chow,
while LF diets consisted of 10.5% kcal/g. The sources of fat
were coconut or corn oil, which are high in saturated fat
and omega-6 polyunsaturated fatty
acids, respectively. “5010” is a standard stock diet.

| Diet | High Fat | Low Fat | 5010 |
|---|---|---|---|
| Fat (% kcal/g) | 58.0 | 10.5 | 13.5 |
| Protein (% kcal/g) | 16.4 | 16.4 | 27.6 |
| Carbohydrate (% kcal/g) | 25.5 | 73.1 | 59.0 |

Study Design

B6.Apc^{Min/+} and the CCSs (A2.Apc^{Min/+}, A7.Apc^{Min/+}, A9.Apc^{Min/+}, A17.Apc^{Min/+}, and A19.Apc^{Min/+}) along with the corresponding wild-type controls (B6, B6.A2, B6.A7, B6.A9, B6.A17 and B6.A19, respectively) were born and maintained on the standard 5010 diet until 30 days of age. Male mice were genotyped for the APC mutation using DNA that was purified from tail tissue taken at weaning. At 30 days of age, male B6.Apc^{Min/+} or CCSs and wild-type littermates weighing between 15-20 grams were placed on a diet that was either high or low in fat from hydrogenated coconut oil (HF_{Coco} or LF_{Coco}, respectively). B6.Apc^{Min/+} and wild-type littermates followed this same diet study design when testing diets high or low in omega-6 polyunsaturated fatty acids (HF_{corn} or LF_{corn}, respectively). Mice were weighed every other day, starting at 30 days of age. At 90 days of age, after 60 days on the diet, mice were fasted for 12-14 hours, and euthanized by cervical dislocation. Body weight and length (to calculate BMI) as well as epidydimal fat pad mass were measured at the final time point.

Body weight and length were measured to calculate body mass index (BMI). Polyp number and polyp size from the small and large intestine were measured. Polyp frequency varies in different regions of the small intestine, so the small intestine was sectioned into four equal parts for analysis. The regions were labeled SI-1 to SI-4, starting from the duodenum located below stomach (SI-1) to ileum which is located above the cecum (SI-4). The large intestine was excised and analyzed as one region (LI-1). Tissue samples from each region (SI-1 to LI-1) were excised and taken to the histology core for sectioning and staining. Most polyps in the B6.Apc^{Min/+} model are benign adenomas, but it is possible that a high fat diet may affect the progression of tumorigenesis.

### Example 2

The experiments described in this example show the effect of diet versus obesity on intestinal cancer susceptibility. After 60 days on the diet described in Example 1, polyp numbers and sizes in lean and obese CCSs were analyzed. All mice fed the HF_{Coco} diet had a significant increase in polyp number compared to mice fed the LF_{Coco} diet, regardless of susceptibility to diet-induced obesity. The obesity-susceptible A2.Apc^{Min/+} had a 3.6-fold increase in polyp number when fed the HF_{Coco} diet, compared to mice fed the LF_{Coco} diet, which was similar to the 3.6-fold increase observed in B6.Apc^{Min/+} fed the same diet. This same trend was detected in the lean CCSs A7.Apc^{Min/+} and A17.Apc^{Min/+} where a 3.0 and 3.4-fold increase was observed, respectively. These findings suggest that obesity is not crucial for polyp development and puts more emphasis on the importance of dietary effects on disease. If obesity were crucial for polyp development, it would have been expected to see an increase in polyp number in obese and not lean CCSs. Total polyp cross section area (calculated using πr²) was increased in all strains fed the HF_{Coco} compared to the LF_{Coco} diet, suggesting that HF_{Coco} affects polyp initiation and not progression. Food intake was measured and there were no significant differences observed between the strains or between strains fed the HF_{Coco} or LF_{Coco} diets.

Body weights and epididymal fat pad mass were measured in all strains for the duration of the study or after 60 days on the diet, respectively. All strains, with the exception of B6.Apc^{Min/+}, had body weights that corresponded to the background strain. A2.Apc^{Min/+}, an obesity-susceptible strain, was obese after 60 days on the HF_{Coco} diet. A7.Apc^{Min/+} and A17.Apc^{Min/+}, two obesity-resistant strains, remained lean after 60 days on the HF_{Coco} diet. After about 40 days on the HF_{Coco} diet, B6.Apc^{Min/+} became overwhelmed with cancer and began to lose weight. This weight loss was not observed in B6.Apc^{Min/+} fed the LF_{Coco} diet. The reduction in body weight could be attributed to the polyp burden or blood loss from anemia (a secondary condition found in B6.Apc^{Min/+} mice).

Measurements were taken of hemoglobin, hematocrit and total red blood cell count (RBCC), which are diagnostic markers used for anemia in humans. Results show that B6.Apc^{Min/+} had significantly reduced levels of hematocrit, hemoglobin and RBCC regardless of diet. The diagnostic markers for anemia were reduced in B6.Apc^{Min/+} fed the HF_{Coco} and LF_{Coco}, suggesting that diet did not contribute significantly to the anemia phenotype.

### Example 3

To test the effect of high dietary fat on inflammatory mediators and Wnt signaling, male B6.Apc^{Min/+} were fed diets high or low in coconut or corn oil for 30 days. B6.Apc^{Min/+} mice were put on diets high or low in omega-6 polyunsaturated fatty acids (HF_{Corn}, LF_{Corn}) or saturated fat (HF_{Coco}, LF_{Coco}) for 30 days. The HF_{Corn} diet has a 30:1 omega-6 to omega-3 ratio, which closely mimics the ratio seen in most Western diets. At the end of the diet study, wild-type tissue from the intestine was collected from wild-type controls, whereas normal and polyp tissues (sized-matched) were extracted from all B6.Apc^{Min/+} mice. Samples were immediately frozen in liquid nitrogen after extraction and stored in a freezer (−80° C.) until analyzed. Tissues were used to extract protein and RNA using radioimmunoprecipitation assays and the QIAGEN RNeasy Mini Kit, respectively. Wild-type, normal tissue from Apc mutants, and polyp tissue were used to extract mRNA or protein for analysis of COX-2, IL-6, IL-1β, NFκB, and TNF-α using qRT-PCR (SYBR Green, Quanta BioSciences) or the appropriate antibodies for western analysis. Serum and plasma were collected from the orbital sinus to measure circulating levels of IL-6, adiponectin, IL-1β, leptin, insulin, MCP-1, IL-10, and TNF-α using Millipore Multiplexed Biomarker Immunoassays.

After 30 days on the diet study, polyp numbers were significantly increased in the mice fed the HF_{Coco} and HF_{Corn} compared to the mice fed the corresponding LF control diet (LF_{Coco} and LF_{Corn}, respectively). B6.Apc^{Min/+} fed the LF_{Coco} (10.6±1.6) diet had a significant reduction in total polyp number compared to mice fed the HF_{Coco} (83.4±16.1) diet (p<0.001). A similar trend in polyp number was observed in B6.Apc^{Min/+} fed the LF_{Corn} (24.6±1.1) and HF_{Corn} (87.2±8.1)(p<0.001). The fact that these two diets are composed of different fat sources suggests that it may not be the kind of dietary fat that influences polyp development, but the quantity. For each mouse, polyp diameter was measured and used to calculate cross sectional polyp area (πr²). Cross sectional polyp area was increased in B6.Apc^{Min/+} after 30 days on the HF_{Coco} and HF_{Corn} diets.

Tissue taken from size-matched polyps and wild-type tissue from the small intestine were used to extract RNA for expression analysis (qRT-PCR). Expression levels of inflammatory biomarkers (COX-2, TNFα, IL-1β, Ptges2) were normalized to 18S and then compared to a LF control. Polyp tissue expression from B6.Apc^{Min/+} fed the HF_{Coco} and HF_{Corn} were compared to expression levels from polyps from mice fed the LF_{Coco} and LF_{Corn} diets, respectively. Preliminary data show that both diets increase inflammatory biomarkers in tissue of the small intestine.

TNFα is produced mostly by macrophages and is involved in systemic inflammation. Polyps from mice fed the HF_{Coco} diet had a noticeable increase in TNFα and Myc (a known Wnt/β-catenin target gene), with 4- and 2-fold increases in expression, respectively. Polyps from B6.Apc^{Min/+} fed the HF_{Corn} diet had a 3-fold increase in TNFα expression relative to polyps from mice fed the LF_{Corn} diet. Similar trends were observed for COX-2, IL-1β, Ptges2 (Prostaglandin E Synthase) and Myc. Although preliminary, this elevation of Myc expression in the polyps of mice fed HF diets could suggest that diet can influence Wnt signaling and promote growth. Expression levels of some of these factors are slightly increased in wild-type tissue from mice fed the HF_{Corn} diet, suggesting that the inflammatory response can be modestly stimulated by dietary fats, independent of carcinogenesis.

Serum samples were collected from B6.Apc^{Min/+} fed the HF_{Coco}, LF_{Coco}, HF_{Corn} and LF_{Corn} after 30 days on the diet studies and were analyzed using Millipore Multiplex cytokine assays. Insulin, leptin, adiponectin, IL-6, IL-1β, TNFα, IL-10, MCP-1, IGF-1, PAI-1 were measured. After 30 days on the diet studies, adiponectin was significantly reduced in B6.Apc^{Min/+} fed either of the HF diets when compared to their corresponding LF control diet. Adiponectin is an insulin-sensitizing hormone that is secreted by adipocytes and is increased in obese individuals and is inversely associated with insulin resistance. Studies have demonstrated that decreased adiponectin levels in men are associated with increased colon cancer risk. Leptin is important in energy expenditure and appetite suppression. Obese individuals develop a tolerance to leptin, similar to the insulin resistance seen in diabetic patients, resulting in an increase in leptin that is proportional to body weight. Elevated levels of leptin in B6.Apc^{Min/+} fed either of the HF diets were observed, suggesting a possible link between leptin and intestinal carcinogenesis. A modest elevation of leptin was observed in B6 control mice fed the HF_{Coco} or the HF_{Corn} diet, suggesting that leptin may be modulated by dietary fat intake. IL-113 and IL-6 are both potent inflammatory factors. Elevation of IL-1β and IL-6 have been observed in human colon cancer patients as well as individuals suffering from obesity. Elevated IL-1β and IL-6 in B6.Apc^{Min/+} mice fed the HF diet were observed. IL-6 was also elevated in B6.Apc^{Min/+} mice fed LF corresponding control diets, suggesting that IL-6 may be involved in intestinal cancer development, independent of diet.

To test the effect of dietary fat on progression of intestinal polyps, B6.Apc^{Min/+} mice were then put on diets high or low in omega-6 polyunsaturated fatty acids (HF_{Corn}, LF_{Corn}) or saturated fat (HF_{Coco}, LF_{Coco}) for 60 days. The HF_{Corn} diet has a 30:1 omega-6 to omega-3 ratio, which closely mimics the ratio seen in most Western diets. After 60 days on the diet, B6.Apc^{Min/+} fed both high fat diets had an increase in total polyp number. It should be noted that in the mice fed the HF_{Corn} diet had a significant increase in total polyp number (100.4±5.0) compared to mice fed the HF_{Coco} diet (58.2±3.5). Diets high in omega-6 fatty acids (corn oil) or saturated fat (coconut oil) increased polyp number, but dietary omega-6 fatty acids had a stronger effect than diets high in saturated fat. Average total polyp size was compared between mice fed the HF_{Coco} and the HF_{Corn}, but no significant difference was observed. Polyp sizes after 60 days on the diet in mice fed the HF_{Coco} and HF_{Corn} were 1.7±0.002 mm and 1.6±0.04 mm, respectively.

Mice fed the LF_{Coco} and HF_{Coco} diets had similar polyp numbers after 30 and 60 days on the diet study. This suggests that while on this diet, polyp numbers stabilize and reach a maximum number after only 30 days. However, mice fed the HF_{Corn} diet showed a significant increase in polyp number from 30 to 60 days, suggesting that the polyps had not reached a threshold after 30 days as seen in mice fed the coconut oil diets. It is possible that diets high in corn oil have a stronger effect on polyp development, or that mice fed the HF_{Corn} diet develop more polyps that take a longer time to stabilize.

In addition to body weight, epididymal white adipose tissue (WAT) fat pads were weighed at the 60 day time point. Final body weights and fat pad weights were analyzed using a One-Way ANOVA and corrected using Bonferroni's Multiple Comparison Test. Overall body weight was significantly reduced in B6.Apc^{Min/+} on both HF diets. Fat pads weights were also significantly reduced in B6.Apc^{Min/+} (p<0.01) on both the HF_{Corn} and HF_{Coco} diets. The reduction in body weight is most likely due to the polyp burden that resulted after 60 days on the HF diet.

### Example 4

This example describes experiments performed to determine the role played by complement signaling in intestinal carcinogenesis in the B6.ApcMin/+ mouse model.

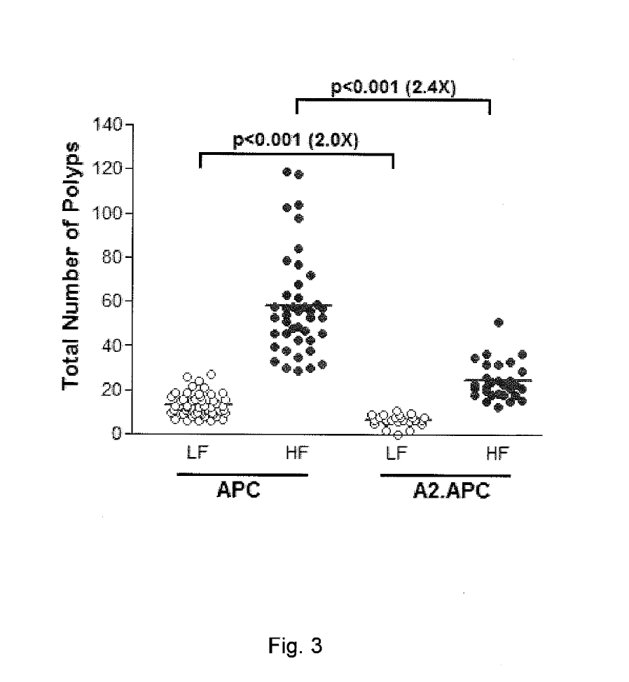

At the end of the 60 day diet study described in the previous Examples, A2.Apc^{Min/+} exhibited a significant reduction in polyp number compared to B6.Apc^{Min/+}, independent of diet, demonstrating the presence of a modifier (FIG. 3). In both strains, a diet high in coconut oil significantly increased polyp number compared to mice fed the low fat control diet, suggesting a dietary effect that functions independent of the modifier.

One candidate gene was the hemolytic component (ortholog of the human complement component C5). Of note, A/J carries a 2-bp deletion in a 5′ exon of the C5 gene that results in a stop codon, rendering them deficient in C5 mRNA and protein production (Wetsel et al. 1990, J. Biol. Chem. 265: 2435-2440).

As described in more detail in Example 1, B6.Apc^{Min/+} were maintained on the standard 5010 diet until 30 days of age. At 30 days of age, male B6.Apc^{Min/+} and wild-type littermates between 15-20 grams were placed on HF_{Coco}, LF_{Coco}, HF_{Corn} or LF_{Corn} diet that is either high (HF) or low (LF) in fat from coconut or corn oil. Mice were weighed throughout the study, starting at 30 days of age. At 90 days of age, after 60 days on the diet, mice were euthanized by cervical dislocation. Serum and plasma samples were collected from the orbital sinus to measure complement component factors as well as other inflammatory mediators. By crossing mice that were deficient in factors involved in innate immunity (C3aR−/−) with mice that carry the B6.Apc^{Min/+}, it was possible to test the influence of dietary fatty acids on different components of complement signaling in colon carcinogenesis.

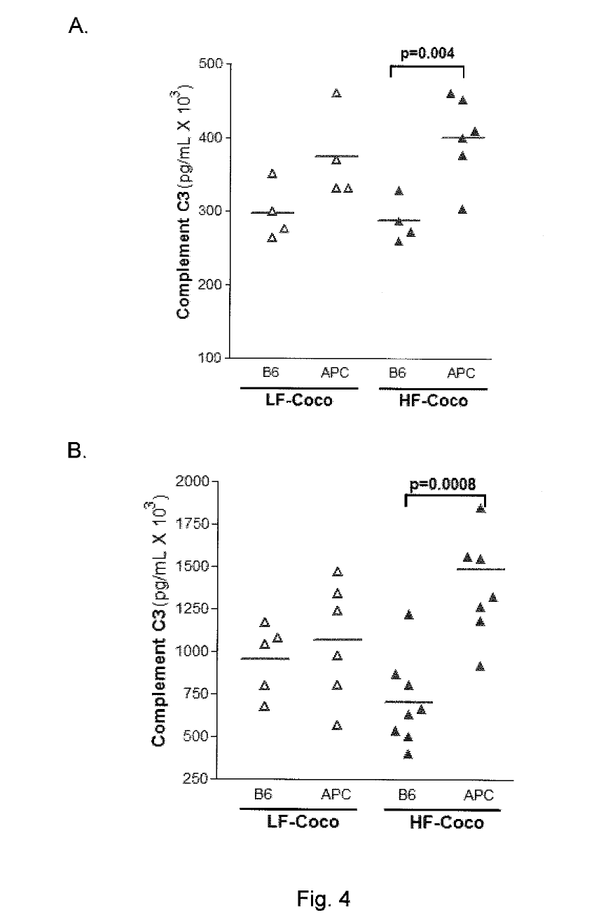

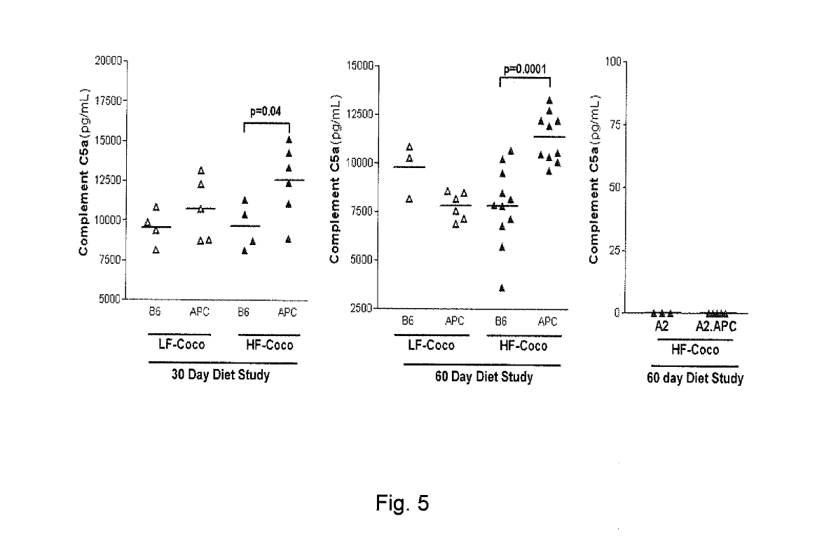

To test if the complement component pathway was activated in the B6.Apc^{Min/+} model of intestinal cancer, circulating levels of C3 and C5a were measured using ELISAs. Complement C3 was elevated in B6.Apc^{Min/+} after 30 (p=0.004) and 60 (p=0.0008) days on the HF_{Coco} diet (FIG. 4). Complement C5a was also elevated in B6.Apc^{Min/+} at these same time points (p=0.04 and p=0.001, respectively) (FIG. 5). Circulating C5a was not detectable in B6.A2 or A2.Apc^{Min/+} after 60 days on the HF_{coco} diet (FIG. 5).

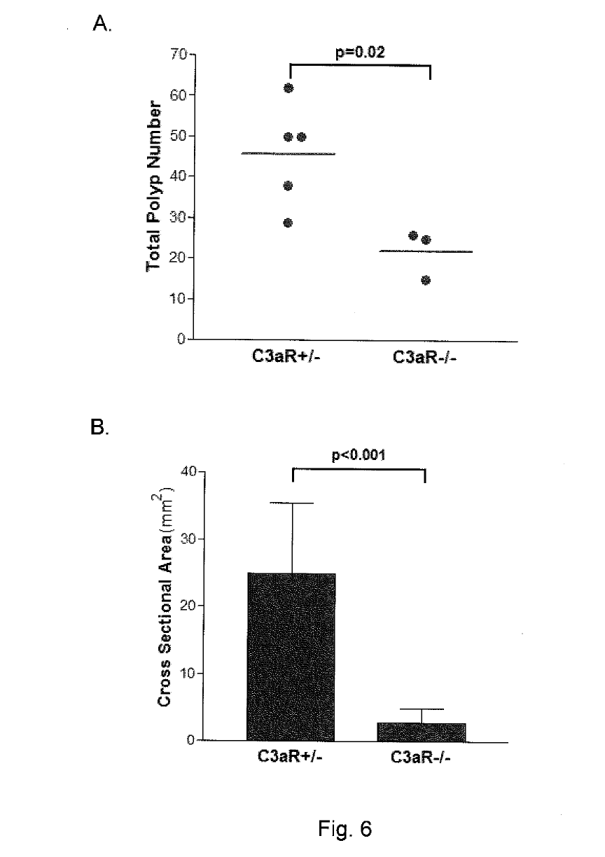

We tested the effect of genetic inhibition of the C3a receptor (C3aR) on polyp number in the B6.Apc^{Min/+} model. After 30 days on the HF_{Coco} diet, double mutant C3aR−/−; Apc^{Min/+} mice exhibited a significant reduction in polyp number compared to mice heterozygous for C3aR, C3aR+/−; Apc^{Min/+} (FIG. 6). Polyp diameter was also measured and used to calculate the cross sectional polyp area for each mouse. A reduction in cross sectional polyp area was also observed in the C3aR−/−; Apc^{Min/+} mice after 30 days on the HF_{Coco} diet.

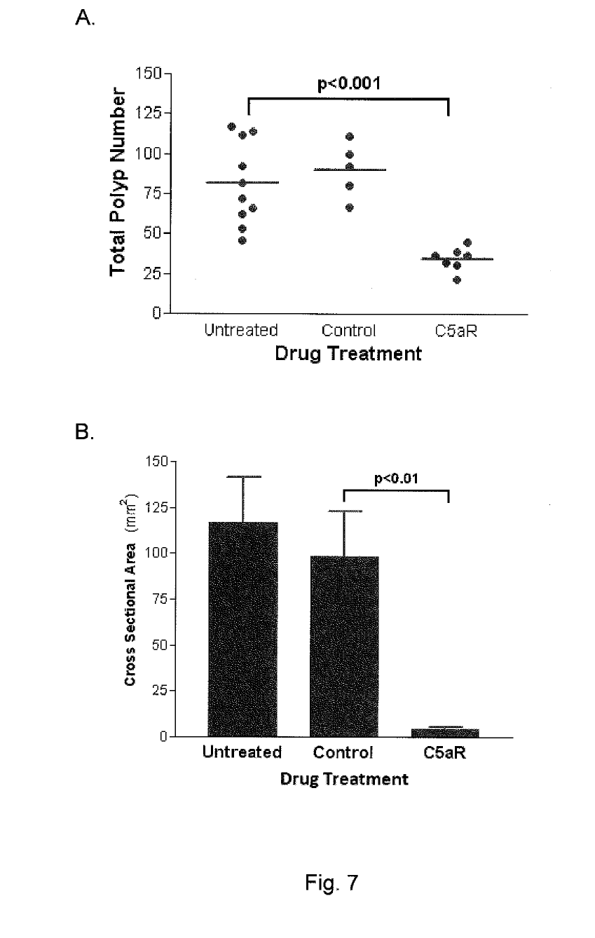

We next tested the effect of ablation of complement factors on the B6.Apc^{Min/+} model, using a pharmacological antagonist for the C5a receptor (C5aR), Acetyl-Phe-[Orn-Pro-D-cyclohexylalanine-Trp-Arg] (AcF[OPdChaWR] (PMX-53; Peptech). Every other day, mice were subcutaneously injected with 1 mg/kg of the C5aR inhibitor or control inhibitor for 30 days. Mice began the drug treatment at 30 days of age, the same day that they began the diet study. After 30 days of treatment, B6.Apc^{Min/+} treated with the C5aR inhibitor had a significant reduction in polyp number compared to untreated mice or mice injected with the control inhibitor (FIG. 7). Cross sectional polyp area was also significantly reduced in B6.Apc^{Min/+} treated with the C5aR inhibitor (FIG. 7).

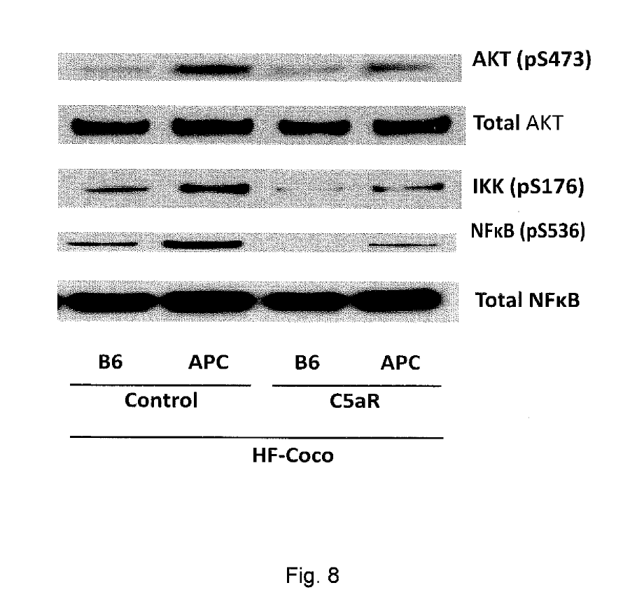

To test the function of this reduction in polyp number in mice treated with the C5aR inhibitor, intestinal tissues were collected and used to extract RNA and protein for expression and western analysis, respectively. After 30 days on the HF_{Coco} diet, mice treated with the C5aR inhibitor has a significant reduction in activated AKT (pS473), IKK (pS176) and NFκB (pS536) protein levels compared to mice treated with the control inhibitor (FIG. 8).

Summary. With the use of the CCSs, a modifier of intestinal neoplasia in the B6.Apc^{Min/+} model was identified. It was shown that complement components C3 and C5a are elevated in B6.Apc^{Min/+} after 30 and 60 days on the HF_{Coco} diet, suggesting an interaction between inflammation mediated by complement signaling and dietary fatty acids.

Deletion of both copies of C3aR(C3aR−/−; Apc^{Min/+}) decreased polyp number and size compared to mice heterozygous for the C3aR KO. Pharmacological inhibition of C5aR also reduced polyp number and size after 30 days of treatment. These results indicate that C5a is modulating polyp initiation and progression in B6.Apc^{Min/+}.

Treatment with the C5aR inhibitor reduced protein levels of activated AKT (pS473), IKK (pS176) and NFκB (pS536) in the intestine of mice fed the HF_{Coco} diet for 30 days. These results point to a role for complement signaling in intestinal neoplasia through the potent anaphylatoxin, C5a, and a point of pharmaceutical intervention targeting the formation or activity of C5a and C5a signaling through the C5aR.

The present invention is not limited to the embodiments described and exemplified above, but is capable of variation and modification within the scope of the appended claims.

1. A method for treating or preventing colon cancer in an individual, the method comprising administering a therapeutically effective amount of a complement inhibitor to the individual, wherein the complement inhibitor reduces or prevents C5a receptor signaling in the tumorigenic or pre-tumorigenic tissue, thereby preventing, reducing or delaying one or more of polyp formation, polyp growth and polyp tumorigenesis.

2. The method of claim 1, wherein the complement inhibitor comprises one or more of a C5a inhibitor, a C5aR inhibitor, a C3 inhibitor, a C3aR inhibitor, a factor D inhibitor, a factor B inhibitor, a C4 inhibitor, a C1q inhibitor, or any combination thereof.

3. The method of claim 2, wherein the complement inhibitor is a C5a inhibitor or a C5aR inhibitor.

4. The method of claim 3, wherein the C5a inhibitor or C5aR inhibitor is acetyl-Phe-[Orn-Pro-D-cyclohexylalanine-Trp-Arg] (PMX-53), PMX-53 analogs, neutrazumab, TNX-558, eculizumab, pexelizumab or ARC1905, or any combination thereof.

5. The method of claim 2, wherein the complement inhibitor is a C3 inhibitor.

6. The method of claim 5, wherein the C3 inhibitor is compstatin, a compstatin analog, or any combinations thereof.

7. (canceled)

8. (canceled)

9. (canceled)

10. The method of claim 1, wherein the complement inhibitor is administered or targeted to the colon.

11. The method of claim 1, wherein the complement inhibitor is administered systemically.

12. The method of claim 1, wherein the complement inhibitor is administered together or concurrently with, or sequentially before or after, at least one other colon cancer treatment.

13. A pharmaceutical composition for the treatment or prevention of colon cancer, said pharmaceutical composition comprising one or more complement inhibitors and at least one colon cancer treatment agent, in a pharmaceutically acceptable medium.

14. The composition of claim 13, wherein the complement inhibitor comprises one or more of a C5a inhibitor, a C5aR inhibitor, a C3 inhibitor, a C3aR inhibitor, a factor D inhibitor, a factor B inhibitor, a C4 inhibitor, a C1q inhibitor, or any combination thereof.

15. The composition of claim 14, wherein the complement inhibitor is a C5a inhibitor or a C5aR inhibitor.

16. The composition of claim 15, wherein the C5a inhibitor or C5aR inhibitor is acetyl-Phe-[Orn-Pro-D-cyclohexylalanine-Trp-Arg] (PMX-53), PMX-53 analogs, neutrazumab, TNX-558, eculizumab, pexelizumab or ARC1905, or any combination thereof.

17. The composition of claim 14, wherein the complement inhibitor is a C3 inhibitor.

18. The composition of claim 17, wherein the C3 inhibitor is compstatin, a compstatin analog, or any combinations thereof.

19. (canceled)

20. (canceled)

21. The composition of claim 13, formulated for administration targeted to the colon.

22. The composition of claim 13, formulated for systemic administration.

23. A method of reducing inflammation in the gastrointestinal system of an individual, the method comprising administering a therapeutically effective amount of a complement inhibitor to the individual, wherein the complement inhibitor reduces or prevents accumulation of one or more proinflammatory molecules in the individual's gastrointestinal system, thereby reducing inflammation.

24. The method of claim 23, comprising reducing inflammation in the colon of the individual.

25. The method of claim 23, wherein the proinflammatory molecules are activated AKT, IKK, NFκB or any combination thereof.


SEQUENCE LISTING (4 sequences)

SEQ ID NO: 1 - protein, 13 aa, Artificial sequence
  synthetic sequence
ICVVQDWGHHRCT

SEQ ID NO: 2 - protein, 16 aa, Artificial Sequence
  synthetic sequence
XXCVXQDWGXHRCXXX

SEQ ID NO: 3 - protein, 16 aa, Artificial Sequence
  synthetic sequence
XXCVXQDXGXHRCXXX

SEQ ID NO: 4 - protein, 13 aa, Artificial sequence
  synthetic sequence
ICVWQDWGAHRCT